(19) United States
(12) Patent Application Publication
(10) Pub. No.: US 2003/0017148 A1
(43) Pub. Date: Jan. 23, 2003

(54) Prevention of insulin-dependent diabetes, complications thereof, or allograft rejection by inhibition of cyclooxygenase-2 activity

(75) Inventors: Tahereh Tabatabaie, Norman, OK (US); Yashige Kotake, Oklahoma City, OK (US)

Correspondence Address: Daniel S. Hodgins, HEAD, JOHNSON & KACHIGIAN, Ste. 3030, 204 N. Robinson, Oklahoma City, OK, 73102, US

(73) Assignee: Oklahoma Medical Research Foundation

(21) Appl. No.: 09/852,587

(22) Filed: May 10, 2001

Related U.S. Application Data
(60) Provisional application No. 60/203,572, filed on May 11, 2000.

(51) Int. Cl. A61K 39/395; A61K 31/415

(52) U.S. Cl. 424/130.1; 514/406

(57) ABSTRACT

Insulin-dependent diabetes mellitus (IDDM) is an autoimmune disease believed to be caused by an inflammatory process in the pancreas leading to selective destruction of the β cells. Inducible cyclooxygenase (COX-2) is expressed under inflammatory conditions and its product prostaglandin E₂ (PGE₂) is an important inflammation mediator. Administration of the selective COX-2 inhibitor such as, e.g., NS-398 prevents the onset of diabetes in mice brought on by multiple low-doses of streptozotocin (STZ). Histological observations indicated that STZ-mediated destruction of β cells was prevented by NS-398 treatment. Delayed (day 3) administration of NS-398 was also protective in this model. These results demonstrate the critical importance of COX-2 activity in autoimmune destruction of β cells, and point to the fact that COX-2 inhibition should provide a preventive therapy against IDDM or other autoimmune problems, including allograft rejection. Inhibitors of NF-κB activation may also be used to prevent IDDM and allograft rejection.

### CROSS-REFERENCED TO RELATED APPLICATIONS

Priority is claimed from provisional application U.S. Serial No. 60/203,572 filed on May 11, 2000, and incorporated by reference herein.

### BACKGROUND

1. Field of the Invention

The present invention relates to a method of prevention of Type 1 or insulin-dependent diabetes mellitus (IDM) by inhibition or repression of inducible enzyme cyclooxygenase (COX-2). IDDM is an autoimmune disease which most often affects children and adolescents. The disease is caused by an attack of the immune system on, and destruction of the pancreatic cells responsible for the production of insulin. This event leaves the body with a lack of ability to make insulin and therefore leads to blood glucose levels which greatly exceed the normal level. Currently there is no cure for this disease, and only available treatment is life-long daily injections of insulin to maintain normal blood glucose level. Tests exist now which can identify the individuals with increased risk of developing Type 1 diabetes (primary candidates are first degree relatives of patients with diabetes). The lack of a cure for this disease plus the possibility of identifying disease candidates make prevention a very attractive and viable alternative. Currently large scale clinical trials to prevent Type 1 diabetes with insulin or nicotinamide are ongoing all over the world. The present results present new therapeutic possibilities in that inhibition of COX-2 activity alone or in combination with other drugs may prevent the onset of diabetes in high risk population.

Insulin dependent diabetes mellitus (IDDM) is an autoimmune disease characterized by local inflammatory reactions in and around the islets of Langerhans in the pancreas. This inflammatory response, also known as insulitis, is caused by infiltration of monocytes, macrophages, and T-lymphocytes with the ultimate consequence of selective destruction of the insulin secreting β-cells in the pancreas (51). Several lines of evidence point to the involvement of the pro-inflammatory cytokines in the pathogenesis of IDDM. In vitro studies have shown that interleukin-1β (IL-1), tumor necrosis factor-α, interferon-β, and particularly the combination of these cytokines impair β-cell function and are cytotoxic to β-cells (6,41,51,58). Administration of a soluble IL-1 receptor protected against diabetes in non-obese diabetic (NOD) mice (45). Similarly, an IL-1 receptor antagonist was shown to prevent the diabetogenic process elicited by the drug streptozotocin (STZ) (57).

Nitric oxide (NO) has also been implicated in the development of IDDM. STZ-induced islet destruction and hyperglycemia in mice was reduced by the nitric oxide synthase (NOS) inhibitor L-N^{G}-monomethylarginine (L-NMMA) (40). Islets isolated from NOD mice were found to produce nitrite (an oxidation product of NO) in vitro (10). Furthermore, selective inhibition of the inducible isoform of NOS (iNOS) was shown to prevent the IL-1-mediated hyperglycemia and hypoinsulinemia in mice (53).

Proinflammatory cytokines induce significant accumulation of prostaglandin E₂ (PGE₂), a key mediator of the inflammatory response which is produced by the enzyme cyclooxygenase (COX), in isolated rat islets (12,25). COX, a key enzyme in arachidonic acid metabolism, converts this compound into PGE₂. There are two different isoforms of COX, the constitutive (COX-1) isoform which is responsible for gastrointestinal and renal protection, and the inducible isoform (COX-2) which is responsible for inflammatory prostaglandin synthesis during tissue injury (15,61). COX-2 is expressed in vitro in response to a number of proinflammatory mediators (i.e. cytokines) and in vivo at the site of inflammation. Corbett et al. have demonstrated that IL-1 induces the expression of COX-2 in the isolated islets (11). Recently, it was reported that there was an increased expression of COX-2 established IDDM patients and in individuals at higher genetic, immunological, and familial risk for this disease (38). Co-expression of COX-2 and iNOS in response to a number of inflammatory stimuli has been documented in both in vivo and in vitro systems (3, 8, 21, 33, 37, 62). Furthermore, it has been shown that expression of iNOS and COX-2 share a common signaling pathway, the activation of the transcription factor nuclear factor-κB (NF-κB) (34, 36). Interestingly, recent studies have shown the existence of a positive feedback loop between the NO and PGE₂ production pathways (9, 47, 50, 63). Therefore, we believe that the positive feedback loop between PGE₂ and NO generation pathways could considerably amplify β cell destruction, and that the inhibition of the PGE₂-producing component of this loop should have a protective role in IDDM development.

A portion of this invention refers to the role of COX-2 inhibition in the prevention or delay of the development of IDDM by using a specific COX-2 repressor or inhibitor such as, e.g., N-(2-cyclohexyl-4-nitrophenyl) methane sulfonamide (NS-398) that possesses specific COX-2 inhibitory activity (IC₅₀ value: 3.8×10⁻⁶ M) in the absence of any effect on COX-1 (19, 20). It is demonstrated herein that the inhibition of COX-2 activity by pharmacological doses of NS-398 results in a near complete prevention of STZ-induced IDDM. These findings point to an important involvement of COX-2 in β cell destruction and open a new avenue in the area of IDDM prevention. Taken into consideration that selective COX-2 inhibitors are widely prescribed for the treatment of rheumatoid arthritis, this investigation suggests that commonly used COX-2 inhibitors can be employed as preventive therapeutics for those genetically predisposed to IDDM.

Streptozotocin (STZ), a methylnitrosourea with a 2-substituted glucose, is widely used to induce experimental insulin-dependent diabetes mellitus (IDDM) in laboratory animals. A single injection of this selective pancreatic β cell toxin causes the induction of IDDM in murine and other rodent models.

In mice, multiple low-dose injection of STZ is widely used for the experimental induction of IDDM. Such treatment has been shown to induce lymphocytic insulitis followed by the onset of autoimmune diabetes (10, 12, 23, 28, 30, 53, 60). In this event, macrophage islet infiltration precedes and presumably helps initiate the lymphocyte response directed at the STZ-induced antigens on β cells (25, 61).

Diabetes is a term that refers to a collection of diseases resulting in disordered energy metabolism and varying degrees of blood glucose elevations or hyperglycemia. One of the best characterized forms of the disease is that which arises from an immunologically mediated destruction of the insulin secreting pancreatic beta cells. This severe form of the disease is termed Insulin-dependent Diabetes Mellitus (IDDM) since it is associated with progressive insulin deficiency and coincident symptoms such as weight loss, glycosuria and polyuria, and increased thirst or polydipsia. Other terms for this form of diabetes are Type 1 Diabetes (cf. Type 2 Diabetes which results from an inherent resistance to insulin action); Ketosis Prone Diabetes because there is abnormal generation of ketone bodies as a result of excessive breakdown of body fats due to the sever insulin deficiency; or Juvenile Diabetes, since virtually all diabetes that appears in childhood and adolescence is of this type.

Diabetes is a major public health problem, especially in Western countries. The incidence rates vary greatly worldwide, from as high as 40 per 100,000 persons in Finland to as low as 1-2 per 100,000 among the Japanese. The peak incidence is during the pubertal years, associated with the increasing bodily demands for insulin associated with muscle growth. The prevalence rates in the United States population under age 20 years in 0.25% and it approaches 0.4% over a lifetime, albeit an estimated 10-20% of patients with Non Insulin-dependent Diabetes Mellitus (NIDDM) or Type 2 or Maturity Onset Diabetes also have, in reality, slowly progressive IDDM. Thus, it is estimated that there may be at least 1 million Americans affected by IDDM.

Diabetes results in progressive damage to the blood vessels of the body, to a degree that depends upon the severity of hyperglycemia and its duration. The incident mortality rate for IDDM has been calculated to be 7-fold higher than for age matched non-diabetic controls. Whereas the decade long Diabetes Control and Complications Trial (DCCT)-concluded in 1994 by the National Institutes of Health in the United States-showed that meticulous insulin replacement therapy would slow the appearance of damaged arteries, it was not able to completely prevent this damage since blood glucose levels were difficult to keep within normal limits. Ocular complications of diabetes are the leading cause of new blindness in persons 20-74 years of age. The risk of lower extremity amputation is 15-fold higher in those with diabetes. Approximately 40% of persons undergoing renal transplants have kidney failure because of diabetes, and the proportion due to diabetes continues to rise each year. Women with diabetes produce newborn infants with a 7% newborn mortality rate. Other complications of diabetes include increased heart disease and stroke, loss of nerve cells or neurones innervating the limbs and intestine, impotence and infertility, cataract formation in the lens of the eyes, increased periodontal disease, and predisposition to infectious diseases especially from bacteria and yeast. Of all patients with diabetes, those with IDDM have a disproportionate share of these complications because of its severity and usual early age of onset. In the Unites States, the direct health care costs attributable to diabetes in 1994 have been estimated to exceed $120 billion. Thus it is important that the pathogenesis of IDDM be understood and strategies be developed to prevent it as a fully expressed clinical disease.

Patients with IDDM are unusually prone to other diseases that have become recognized as having autoimmune origins. These diseases include thyroiditis or Hashimoto's disease, Graves' disease, Addison's disease, atrophic gastritis, pernicious anemia, celiac disease, and vitiligo (Maclaren, N. K. [1985] Diabetes Care 8(suppl.):34-38). Evidence that IDDM itself has an autoimmune nature began with histological studies of patients; these studies indicated that the islets were infiltrated with a chronic inflammatory (lymphocytic) infiltrate termed insulitis. This was supported in the early 1970s by reports of islet cell autoantibodies reactive to antigens within the cytoplasm (ICA) (Lendrum et al. [1975] Lancet 1:880-882) or confined to the islet cell surfaces (ICSA) (Maclaren et al. [1975] Lancet 1:977-1000) as detectable by indirect immunofluorescence. Later it was recognized that many patients also develop autoantibodies to insulin (IAA) before their diagnosis(Palmer et al. [1983] Science 222:1337-1339) as well as to insulin receptors (Maron et al. [1983] Nature 303:817-818). Autoantibodies were also reported to anislet cell protein composition of 64,000 M.Wt. in man (Baekkeskov et al. [1982 Nature 298:167-169), in the Biobreeding (BB) rat model (Baekkeskov et al. [1984] Science 224:1348-1350), and in the Non Obese Diabetic (NOD) mouse model (Atkinson and Maclaren [1988] Diabetes 37:1587:1590). 64 kDa antigen has subsequently been reported to be the lower molecular weight isoform of glutamic acid decarboxylase (GAD₆₅) (Kauffman et al. [1992] J. Clin. Invest. 283-292). GAD is an enzyme that converts glutamate into the membrane stabilizing neurotransmitter called gamma amino butyric acid or GABA. In addition to autoantibodies to GAD, peripheral blood mono-nuclear cells were shown to be autoreactive in patients developing IDDM (Atkinson and Maclaren et al. [1992] Lancet 339:458-459; and Harrison et al. [1993] Lancet 341:1365-1369).

Recently issued U.S. Pat. No. 6,048,850 (which is incorporated by reference herein), describes cyclooxygenase-2 (also known as prostaglandin H synthase-2) its isolation and therapeutic values of inhibition.

### SUMMARY OF THE INVENTION

IDDM is characterized by an inflammatory reaction in and around the islets of Langerhans of the pancreas which leads to selective destruction of the insulin-producing β cells. The enzyme COX-2 forms prostaglandin E₂ which is a key mediator of the inflammatory response. The present invention demonstrates that inhibition of COX-2, by inhibiting the expression of COX-2 or activation of NF-κB by, e.g., PDTC and/or a selective inhibitor of this enzyme, such as NS-398, prevents the development of IDDM in mice treated with the diabetogenic compound, streptozotocin (STZ). Other inhibitors of NF-κB activation such as PBN (phenyl N-tert-Butylnitrone) may be likewise to inhibit expression of COX-2 and the like (64). This conclusion was reached based on the measurement of the blood glucose level, glycated hemoglobin level (a marker of chronic hyperglycemia) and by histological examination of the islet morphology. All these parameters were close or indistinguishable from normal in mice receiving STZ and NS-398 or STZ and PDTC, whereas mice treated with STZ alone showed all expected diabetic symptoms. These results demonstrate that COX-2 activity inhibition or repression represents an approach to prevention of IDDM in at-risk individuals.

Diabetes is associated with long-term complications including retinopathy, neuropathy, cardiovascular disease and kidney disease. These complications are believed to be the result of the long-term elevated blood glucose levels which results in the glycation of cellular proteins and the formation of advanced glycation end-products or AGE's. Formation of AGE's has been associated with increased inflammation in the tissues they form in and inflammation may be the cause of the observed complications. Therefore, the anti-inflammatory effect of COX-2 repression or inhibition will be effective in preventing the complications of diabetes as well.

An important aspect of the present invention is a method for the treatment of diabetic complications comprising administering an effective amount of cyclooxygenase-2 inhibitor or repressor of NF-κB activation. Such diabetic complications include nephropathy, retinopathy, neuropathy, and cardiovascular disease. The use of cyclooxygenase-2 inhibitors or NF-κB repressors do both help in maintaining normal glucose levels as well as to prevent abnormal inflammatory effects, will alleviate at least to a great extent many of these complications. Again a preferred COX-2 inhibitor is NS-398, and a preferred repressor of NF-κB activation is PDTC. Therapeutic modalities would be about the same as those for the treatment of prevention of Type 1 diabetes.

Individuals with an immediate family history of Type 1 diabetes are at risk for IDDM and would benefit from therapy with COX-2 repressors or inhibitors. For individuals with such a family history, screening for anti-pancreatic beta cell-directed antibodies should be performed. For those showing signs of these antibodies, immediate initiation of therapy with such COX-2 specific inhibitors or repressors is warranted to prevent frank Type 1 diabetes development. Appropriate inhibitors doses should be from about 2 mg/kg body weight to about 10 mg/kg body weight. Of course those inhibitors and/or repressors with lower IC₅₀ values are needed in lower amounts. Drug administration may be enteral or parenteral, whichever allows for effective results.

COX-2 inhibitors and/or repressors of NF-κB activation may also be utilized for the prevention of allograft rejections. PGE₂ elevation has been observed in pancreas allograft rejection. Elevated PGE₂ level has been suggested as an early marker of pancreas transplant rejection. Thus, prevention of excessive PGE₂ formation by means of NS-398 or other selective COX-2 inhibitors or repressions of NF-κB activation will be of value in prevention of islet transplant rejection in diabetic patients. There is some evidence that the extent of PGE₂ release in urine can act as a predictor of kidney transplant rejection (the higher the PGE₂ level, the higher the chance of rejection). Furthermore, an increase in arterial and portal blood PGE₂ level has also been observed in liver transplantations and, while this level returned to normal with un-rejected livers, it did not do so with rejected livers. Thus, PGE₂ production appears to contribute to morphological and functional alterations of the transplanted graft Also, upregulation of COX-2 during cardiac allograft rejection has also been observed. Thus, inhibition of COX-2 expression and/or activity and PGE₂ formation will increase the chance of graft survival. For example, Zheng, et al (A. M. Surg. 1992 October; 58(10):630-3) indicated that elevated urinary prostaglandin-2 (PGE-2) increased after the allograft pancreatic transplantation (also see Zheng, et al. Transplant. Proc. 1990 APR:22 (2): 732-3 and Soon-Shiong, et al., Transplant. Proc. 1989, February 21(1 PT 3): 2771-3). Analogous observations have been made for allografted kidneys. See Coffman, et al. Kidney Int. 1989 January, 35(1) 24-30; Mangino, et al. J.Pharmacol. Exp. Ther. 1989 January, 248(1) 23-8; Coffman, et al, Transplantation 1988 February, 45(2) 470-4; Ladefoged, Prostaglandins 1987 October, 34(4) 611-8; Tannenbaum, et al. Transplantation 1984 May, 37 (5) 438-43; Thompson, et al. Ann. Clin. Biochem. 1985 March, 22 (Pt 2) 161-5. Liver allografts have given rise to similar observations: Post, et al. Transplantation 1991 May, 51 (5) 1058-65; Post, et al. Transplantation 1990 March, 49(3) 490-4. In at least one instance, cardiac allograft rejection has resulted in similar events: Yang, et al. Circulation, 2000 February, 1101(4) 430-8.

Thus COX-2 Inhibitors may be administered to recipients or prospective recipients of various allografts in order to inhibit the rejection of such allografts.

A method for prevention of insulin-dependent diabetes comprising administering an effective amount of a cyclooxygenase-2 inhibitor or repressor of NF-κB activation to an individual having a possible predisposition to or showing signs of development of Type 1 diabetes.

A preferred cyclooxygenase-2 inhibitor is NS-398.

A preferred repressor of NF-κB activation is PDTC.

The preferred therapeutic effective amount of cyclooxygenase-2 inhibitor is from about 2 mg/kg to about 5 mg/kg body weight, administered enterally or parenterally.

The preferred effective amount of repressor such as PDTC is from about 25 to about 250 mg/kg/day, administered enterally or more preferably parenterally.

An individual's predisposition is determined by at least one of: a history of Type 1 diabetes in an immediate family member and/or finding antibodies directed toward antigens of the individual's pancreatic beta cells.

Allograft rejections may also be inhibited comprising administration of an effective amount of a COX-2 inhibitor or repressor NF-κB to an allograft recipient.

Such an allograft includes, e.g., pancreas, kidney, liver and heart.

### BRIEF DESCRIPTION OF THE DRAWINGS

FIG. 1 shows the effect of NS-398 administration on STZ-induced hyperglycemia. Mice were treated with PBS (▴); STZ, 40 mg/Kg (▪); or STZ, 40 mg/Kg+NS-398 10 mg/Kg (●) for the noted number of days. Values are reported as the mean±SD (n=10). * and ** Denote statistically significant differences between STZ and STZ+NS-398 treated groups (*: p<0.05, **: p<0.01).

FIG. 2 shows the effect of NS-398 treatment on STZ-induced increase in % glycated hemoglobin, a marker of chronic hyperglycemia. * Denotes statistically significant differences between STZ and STZ+NS-398 treated groups (p<0.05).

FIGS. 3A-3C show mouse pancreatic tissue slices stained with aldehyde fuchsia.

FIG. 3A: Control (untreated) pancreas. An islet with intact β cells (dark cells within the islet) is shown.

FIG. 3B: STZ-treated mouse pancreatic tissue. β cells contained within the islets are virtually absent in STZ-treated pancreas.

FIG. 3C: STZ+NS-398 treated pancreatic tissue. In these tissues, a majority of β cells are preserved.

FIG. 4 shows DFU Analogs containing an oxygen link between the O-Fluorophenyl and the lactose ring of DFU.

FIG. 5 shows 2-alkoxy, thiolalkoxy, and 2-amino-3-(4-methyl sulfonyl) phenyl pyridine (Merck Frosst).

FIG. 6 shows sulfonyl-substituted 4,5-diarylthiazoles.

FIG. 7 shows sulfonamide-substituted 4,5-diaryl thiazoles (GD-Searle-Monsanto). Most selective and potent compound of this series: R₁=H—, R₂=4—SO₂NH₂, R₃=—CH₃.

FIG. 8 shows 5-Keto-substituted 7-tert-butyl-2,3-dihydro-3,3-dimethyl benzofurans (Proctor & Gamble). Most promising isomer: R=CO(CH₂)₃-c—Pr.

FIG. 9 shows 3,4-diaryloxazolones (Almirall Prodesfarma, Spain).

FIG. 10 shows 2,3-Diarylcyclopentenones (Merck Frosst)

L-776,967 (ED₅₀: mg/kg) R=3,5-diflurophenyl

L-784,506 (ED₅₀: 1.7 mg/kg R=3-pyridyl.

FIG. 11 shows 4,5-Diaryloxazoles (G.D. Searle-Monsanto) (methyl sulfonyl or sulfonamido substituted).

FIG. 12 shows Ditert-butyl phenols (1,3,4-and 1,2,4-thiadiazole series) (Parke-Davis).

FIG. 13 shows Diarylspiro [2.4] heptenes (G.D. Searle/Monsanto).

FIG. 14 shows Terphenyl methyl sulfones and sulfonamides (Searle/Monsanto) 1,2-diaryl-4,5-difluorobenzene sulfonamide.

FIG. 15 shows Indolealkanoic acids (Merck-Frosst).

FIG. 16 shows 1,2-Diarylcyclopentenes (Searle/Monsanto).

FIG. 17 shows 5-Methane sulfanamido-1-indanone derivatives.

FIG. 18 shows the isoprelated inhibitor: Flosulide.

FIG. 19 shows 3-Heteroaryloxy-4-phenyl-2(5H)-furanones (Merck Frosst).

FIG. 20 shows substituted heterocyclic analogs in the Flosulide class (Merck Frosst).

FIG. 21 shows 2-benzyl-4-sulfonyl-4-H-isoquinoline-1,3-diones (Boehringer Ingelheim Pharm.)

FIG. 22 shows 2-pyridinyl-3-(4-methylsulfonyl) phenylpyridines (Merck Frosst).

FIG. 23 shows Di-tert-butylphenols (thiazolone and oxazolone derivative) (Parke-Davis).

### DETAILED DESCRIPTION OF THE PREFERRED EMBODIMENTS

### EXAMPLE 1

### Cox-2 Inhibitors

Materials and Methods

Chemicals: Streptozotocin was obtained from Sigma Chemical Company (St. Louis, Mo.). NS-398 was obtained from Cayman Chemicals (Ann Arbor, Mich.). Determination of blood glucose level was performed using the glucose oxidase kit obtained from Sigma Chemical Co. Total glycated hemoglobin (Hb A₁) was measured using a kit from Sigma.

Animal treatment: Ball/c or CD-1 mice (male, 6-8 weeks) from Charles-River (Indianapolis, Ind.) were used in this study. STZ was dissolved in citrate buffer pH 4.5 and was administered within 10 minutes of its dissolution. Animals receiving NS-398 treatment were injected with this compound 15 min prior to STZ treatment. Since STZ is both toxic and carcinogenic, extreme caution was practiced in its handling. NS-398 was dissolved in dimethylsulfoxide (DMSO). Injection volume for STZ and NS-398 were 200 μl and 40 μl, respectively. To control for the effect of DMSO, mice in the STZ group received 40 μl injections of DMSO for the same number of days as the STZ+NS-398 group. At different time intervals after the treatments, tail tip blood was drawn and used for the measurement of blood glucose levels and/or glycated hemoglobin. Treatment regimen for different experiments are summarized in Table I below.

TABLE I

| | | | COX-2 Inhibitor | |
|---|---|---|---|---|
| Experiment | STZ | | Start | Total # of |
| # | Treatment | (dose/day) | Date | Injections |
| 1 | 5-day, | NS-398 | day 1 | 11 |
| | 40 mg/Kg/day | (10 mg/Kg) | | |
| 2 | 5-day, | NS-398 | Group 1: day 1 | 11 |
| | 40 mg/Kg/day | (5 mg/Kg) | Group 2: day 3 | 9 |
| 3 | 1 single injection | NS-398 | same day | 1 |
| | 220 mg/Kg | (10 mg/Kg) | | |
| 4 | 5-day, | indomethacin | day 1 | 5 |
| | 40 mg/Kg/day | (3 mg/Kg) | | |

As listed in the above Table I, in experiment 1, mice (10 mice per group) were given daily intraperitoneal (ip.) injections of: STZ, 40 mg/Kg/day (STZ group); STZ, 40 mg/Kg/day and NS-398, 10 mg/Kg/day (STZ+NS-398 group); PBS (control group) for 5 consecutive days. For this group, NS-398 injection continued for 6 days following the conclusion of STZ treatment. Blood glucose level for these animals was determined periodically for a duration of 30 days following treatment. In experiment 2, the effect of delayed NS-398 administration was studied. For this purpose, three groups of mice (5 mice/group) were treated with 5 daily STZ doses (40 mg/Kg/day). To the first group of these mice (STZ+NS-398 group) NS-398 was administered daily (5 mg/Kg, ip. injection) starting at day 1 of STZ treatment and continued for 6 extra days following the completion of STZ treatments. For the second group (STZ+Delayed NS-398), NS-398 treatment was started at day 3 of STZ injections and continued for 6 extra days following the completion of STZ treatments. The STZ group received only STZ injections. In experiment 3, the effect of NS-398 on the single high-dose STZ-induced IDDM was determined. Twelve mice were given a single injection of a high dose (220 mg/Kg) of this compound. In this set of experiments, NS-398 (10 mg/Kg) was pre-administered to half of these animals (n=6),15 min prior to STZ treatment. Blood glucose was determined in these animals 48 h following the treatment. In experiment 4, two groups of mice (5 mice/group) were treated with STZ (40 mg/Kg/day for 5 days). One group of these mice received daily treatments of indomethacin (3 mg/Kg/day). Indomethacin was dissolved in DMSO and an injection volume of 40 μl was used.

For histological studies, 35 days following the treatments (multiple low-dose STZ±NS-398), mice were anaesthetized and perfused with 4% paraformaldehyde and pancreata were removed and post-fixed for an additional day. The paraffin sections (6 μ) were stained with Gambier's aldehyde fuchsia for the identification of pancreatic β cells. Untreated mice were used as controls for these studies.

Statistical analyses: Statistical analyses of the data were performed using one way analysis of variance (Anoa) followed by Student's t-test.

In order to determine whether NS-398 treatment would protect the animals against the diabetogenic action of STZ, blood glucose level, and the level of glycated hemoglobin (a marker of chronic hyperglycemia) were monitored in the mice treated with STZ in the absence and presence of NS-398. Histological studies were also performed on the pancreata of the treated animals in order to obtain morphological information on the status of the islet β cells.

### NS-398 PREVENTION OF THE STZ-INDUCED RISE IN BLOOD GLUCOSE LEVEL OF THE TREATED ANIMALS

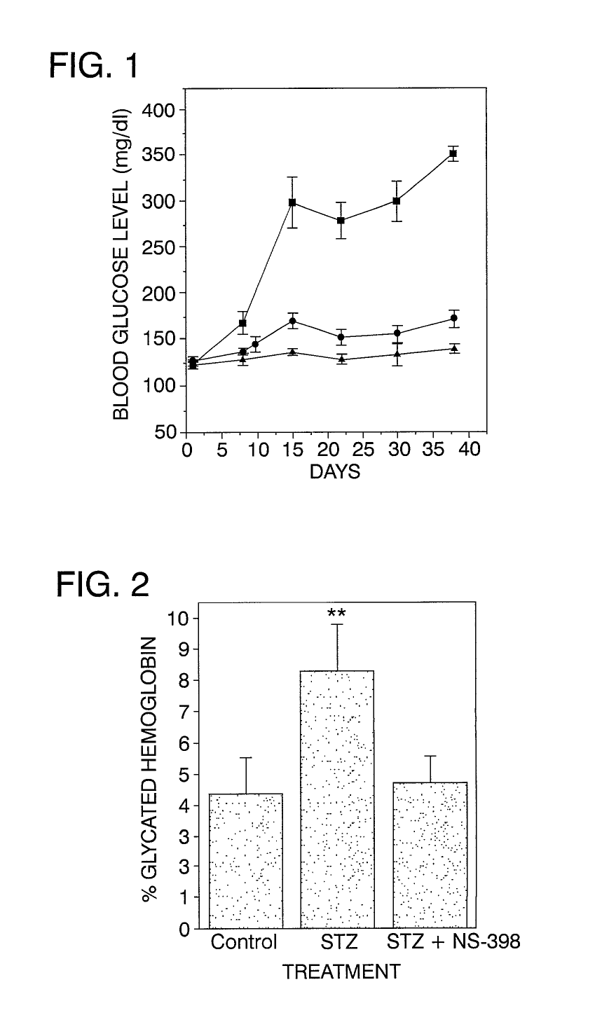

Mice treated with STZ demonstrated a progressive rise in the blood glucose concentration. The blood glucose level of the animals which received NS-398 treatment, however, stayed near normal throughout the experiment, during the course of NS-398 administration and for up to 28 days after the cessation of NS-398 treatment (FIG. 1). The differences between the two groups (STZ vs. STZ+NS-398) were statistically significant (p<0.05 at days 15, 22, 30, and p<0.001 at day 38). Control mice (untreated or treated with PBS) displayed blood glucose levels within the normal range throughout the experiment. Similar results were obtained when CD-1 mice were used in the study.

### PREVENTION OF THE STZ-INDUCED INCREASE IN THE GLYCATED HEMOGLOBIN LEVEL BY NS-398 PRETREATMENT

The STZ-treated mice demonstrated significantly elevated levels of glycated hemoglobin 30 days following the treatment (p<0.05). However, the mice which received NS-398 treatment, exhibited glycated hemoglobin values in the same range as the control mice. As expected, the control mice had low levels of glycated hemoglobin (FIG. 2).

### PROTECTIVE EFFECT OF DELAYED ADMINISTRATION OF NS-398 AGAINST STZ-INDUCED IDDM

Delayed NS-398 treatment (starting at day 3 of the 5-day STZ treatment) was still effective in protecting the treated mice from STZ-induced diabetes (Table II). There were no statistically significant differences between the blood glucose level of the mice receiving NS-398 from the beginning of the experiment and those which were given NS-398 starting at the third day of STZ treatment. Furthermore, the fact that both these groups showed a significant degree of protection against IDDM in spite of the shorter duration of NS-398 treatment indicates that COX-2 inhibition at the early stages of STZ-induced β cell destruction, was able to effectively curb the magnitude of these events. CD-1 mice showed similar results.

TABLE II
Effect of delayed NS-398 treatment on STZ-induced diabetes

| Treatment | Blood Glucose Level (mg/dl)* | n |
|---|---|---|
| STZ | 304.0 ± 91.6** | 5 |
| STZ + NS − 398 | 151.2 ± 26.7 | 5 |
| STZ + Delayed NS − 398*** | 161.8 ± 17.4 | 4 |

*These values were obtained 30 days following the completion of STZ treatment. Results are expressed as mean± standard deviation. **There was statistically significant difference between the STZ and the two NS-398-treated groups (p<0.02). No statistically significant difference was observed between STZ+NS-398 and STZ+Delayed NS-398 groups. ***NS-398 treatment was started at day 3 of the 5-day STZ administration schedule. For both groups, NS-398 administration was continued 6 days following the completion of STZ treatment.

### INEFFECTIVENESS OF NS-398 AGAINST HIGH-DOSE STZ-INDUCED IDDM

NS-398 did not protect against the diabetogenic effect of one single high dose of STZ. Mice pre-treated with this compound all displayed hyperglycemia as a result of STZ treatment (Table III).

TABLE III
Effect of NS-398 pre-treatment on high-dose STZ-induced
hyperglycemia

| Treatment | Blood Glucose Level (mg/dl)* | n |
|---|---|---|
| STZ (220 mg/Kg) | 311.1 ± 80.7** | 6 |
| STZ + NS − 398 | 348.8 ± 39.1 | 6 |

### EFFECTS OF THE NON-SELECTIVE COX INHIBITOR INDOMETHACIN

The non-selective inhibitor of COX enzymes, indomethacin was not well tolerated by the mice even at the moderate dose of 3 mg/Kg (55). Four out of five mice treated with indomethacin died during or following the completion of the treatment. Obviously we were not able to obtain any positive results from these experiments. Even though we did not perform necropsy on these animals, stomach ulceration is the most probable cause of death in these animals (55).

### HISTOLOGICAL STUDIES

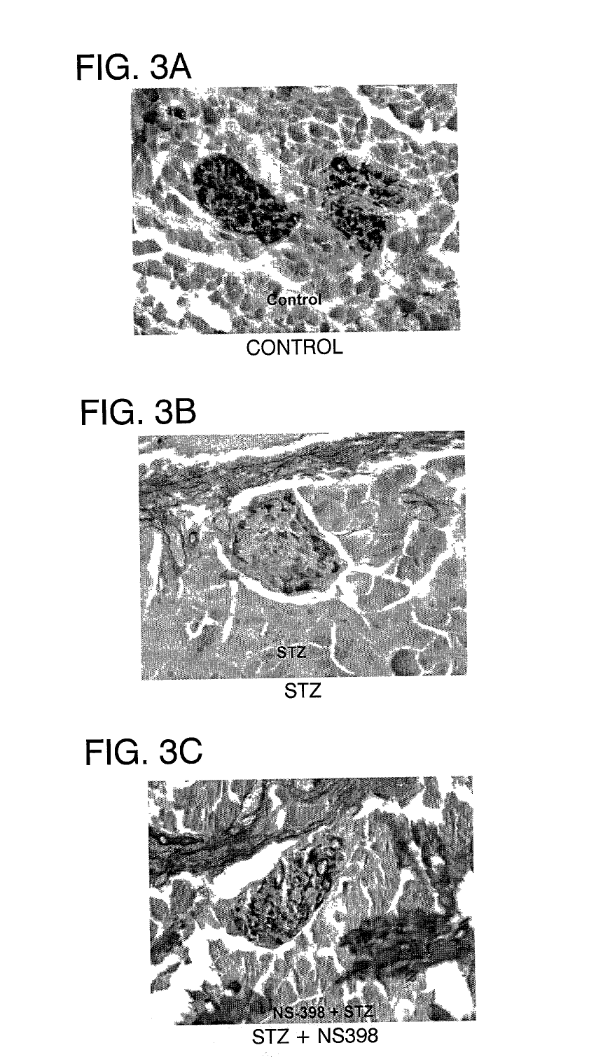

Histological inspection of pancreatic tissue indicated that STZ treatment had caused the destruction of pancreatic β cells (FIG. 3B). The β cells (stained with aldehyde fuchsin and seen as darker spots within the islets in the figures) were nearly completely destroyed as a result of STZ treatment. In mice treated with STZ+NS-398 however, the majority of β cells within the islets appeared to be well preserved (FIG. 3C). These results clearly indicate that the STZ-induced destruction of the islets β cells was prevented by NS-398 in this tissue.

This study demonstrates for the first time that selective inhibition of COX-2 protects against the onset of STZ-induced IDDM in mice. Pre-treatment with selective COX-2 inhibitor NS-398 significantly inhibited STZ-induced rise in blood glucose level, while the STZ group showed clear hyperglycemia (FIG. 1). In agreement with the observed blood glucose concentrations, the level of glycated hemoglobin was almost doubled in the STZ group, whereas animals that had been treated with STZ+NS-398 showed normal glycated hemoglobin levels (FIG. 2). Histological investigation of the pancreatic tissue of these animals revealed that islet β cells were nearly all destroyed in STZ treated animals (FIG. 3B). In the STZ+NS-398 group, while the number of β cells were decreased as compared to the control tissues, a majority of these cells were preserved. These data clearly demonstrate a protective role for NS-398 against STZ-induced IDDM and therefore, point to the critical role of COX-2 in the processes leading to islet cell damage.

COX is the rate-limiting enzyme in prostaglandin and thromboxane synthesis pathway (20). The inducible isoform of this enzyme, COX-2, plays a central role in mediating inflammatory events. COX-2 is expressed under inflammatory conditions, and converts arachidonic acid into the inflammatory mediator PGE₂. Inhibition of COX-2 activity by means of its selective inhibitors has been effective in ameliorating chronic inflammatory diseases such as rheumatoid arthritis (63). It is well documented that cytokine exposure leads to the induction of COX-2 in various in vitro systems (11, 26, 62). In isolated rat islets, the cytokine IL-1 promotes COX-2 expression and PGE₂ formation (11, 12, 25). In other studies, pro-inflammatory cytokines have been reported to cause the co-expression of iNOS and COX-2 indifferent systems including isolatedrat islets (3, 8, 11, 21, 33, 37, 62). Cytokine-induced expression of iNOS and the ensuing NO over-production by β cells as well as infiltrating macrophages, have been shown to be instrumental in the destruction of β cells. Indeed, induction of iNOS has been demonstrated in the pancreas of NOD mice and BB rats (31, 52, 68). Transgenic mice expressing iNOS in their β cells were shown to develop IDDM without going through an insulitis phase (65). In addition, it is now clear that interaction between PGE₂ and NO pathways enhances the expression of COX-2 in the inflamed tissue (9, 47, 50, 69). Nogawa et al. have reported that in a focal cerebral ischemia model, a selective iNOS inhibitor aminoguanidine, reduced PGE₂ formation in the infarct area (46). In this study, the authors showed that in iNOS-deficient mice, post-ischemic PGE₂ accumulation was significantly diminished. In the same model of ischemic-reperfusion injury, administration of NS-398 was found to reduce the infarct volume and to ameliorate the ischemia-induced motor neuron deficits in wild-type mice but not in iNOS-deficient mice (44). McDaniel et al. have suggested that one reason for increased PGE₂ formation in the presence of NO is that NO may directly activate COX-2 through its interaction with the heme moiety of this enzyme (43).

These observations taken together with the well-documented role of iNOS in IDDM pathogenesis prompted us to investigate the role of COX-2 in the destruction of β cells in low-dose STZ-induced IDDM. In this model of autoimmune diabetes, NOS inhibitors, L-N-nitroarginine-methylester (L-NAME) and L-NMMA were shown to protect from diabetes, thus implicating NO in STZ-mediated β cell destruction (32, 40). Moreover, Flodstrom et al. recently demonstrated that iNOS-knockout mice exhibit diminished sensitivity to multiple low-dose STZ-induced IDDM, therefore confirming a role for iNOS-mediated generation of NO in this model (17). Our observations demonstrate that inhibition of COX-2 activity in the initial stages of the events leading to β cell death is effective in halting the attack on these cells (Table II). This clearly indicates that COX-2 plays a pivotal role in the initiation and propagation of inflammatory processes. In addition, the effectiveness of the delayed NS-398 treatment in protecting against STZ-induced IDDM, demonstrates that the inhibition of COX-2 activity arrests or cuts off the β cell-destroying inflammatory cascades even after their initiation.

Previous studies have shown that PGE₂ plays a promotional role in inflammation through induction of yet other inflammatory factors. For example, PGE₂ has been shown to be a critical regulator of IL-6 production (3, 24). Selective inhibition of COX-2 or neutralization of PGE₂ by means of its monoclonal antibody blocked inflammation and IL-6 production in two different models of synovial inflammation (1, 49). Administration of IL-6 antibody caused reduced severity of insulitis and a lower incidence of autoimmune diabetes in NOD mice (7), thus pointing to the important role of IL-6 in the inflammatory events preceding β cell damage and death. COX-2 inhibition has been previously shown to diminish the level of infiltrating monocytes and COX-2 MRNA in arthritic joints (1). Also, of possible significance to the findings of this study is the observation that inhibition of COX-2 by means of nonsteroidal anti-inflammatory drugs (NSAIDs), suppresses the production of phospholipase A₂ (PLA₂) mRNA and thus deprives COX-2 from its substrate arachidonic acid, released from phospholipids by PLA₂, indicating that PGE₂ is involved in the regulation of PLA₂ expression (70). Furthermore, Baek et al. have shown that inhibition of PLA₂ leads to a down-regulation of lipopolysaccharide-induced expression of iNOS, COX-2 and TNF-α proteins (2). All these evidence point to the potentiating effect of PGE₂ formation on the inflammatory response, and thus may explain the lasting effect of COX-2 inhibition on β cell protection in the employed model. The lack of effect of NS-398 against high-dose STZ-induced IDDM (Table III), confirms the notion that protective effect of this compound is due to the interruption of inflammatory events set in action by multiple low-dose STZ (37), and not due to counteracting STZ's direct toxic effect.

Autoimmune diabetes can now be diagnosed in its early, pre-symptomatic stages. Islet cell autoantibodies (ICA's) are present in pre-symptomatic stages of the disease, and represent a valuable marker for the autoimmune reaction (66, 59). Thus, it is now possible to identify individuals who are at increased risk of the development of IDDM, and therefore intervention of the disease process has become a logical direction in the area of IDDM research. The data herein presented indicate that COX-2 selective inhibitors which are now widely used in treatment of other inflammatory diseases such as rheumatoid arthritis (22) can potentially serve as preventive therapeuticals for individuals with increased risk for developing IDDM.

Other COX-2 inhibitors usable to prevent Type 1 diabetes and its negative side effects include:

FDA approved:

Celecoxib (Celebrex) (JC58635): sulfonamide-containing 1,5-diarylpyrazole class; and

Rofecoxib (Vioxx)

Others:

a) DFU: 5,5-dimethyl-3-(3-Fluorophenyl)-4-(4-methanesulfonylphenyl)-2(5H)-Furanone. (Merck Frost).

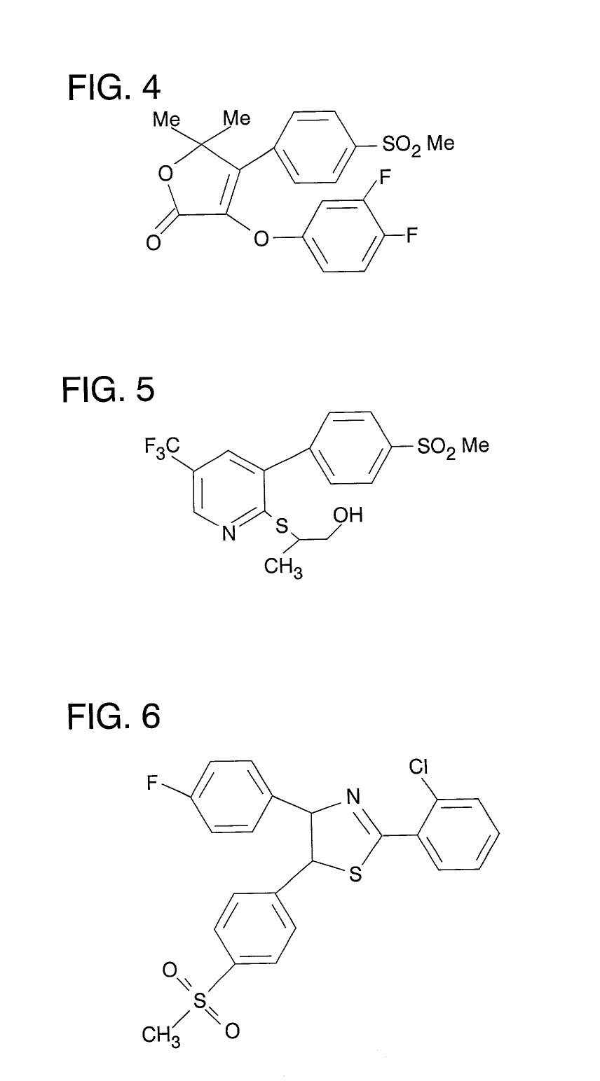

b) DFU Analogs containing an oxygen link between the O-Fluorophenyl and the lactone ring of DFU. ED₅₀ (rat): 0.1-1.3 mg/kg, NO GI toxicity).(See FIG. 4)

c) DuP-697 ED₅₀: 0.18 mg/kg:

d) 2-alkoxy, thiolalkoxy, and 2-amino-3-(4-methyl sulfonyl) phenyl pyrdines (Merck Frosst). Best isomer shown in FIG. 5. (ID₅₀=0.65 mg/kg, ED₅₀=0.3 mg/kg).

e) Sulfonyl-substituted 4,5-diarylthiazoles (ED₅₀=20 mg/kg) (GD-Searle). Best compound in this series shown in FIG. 6.

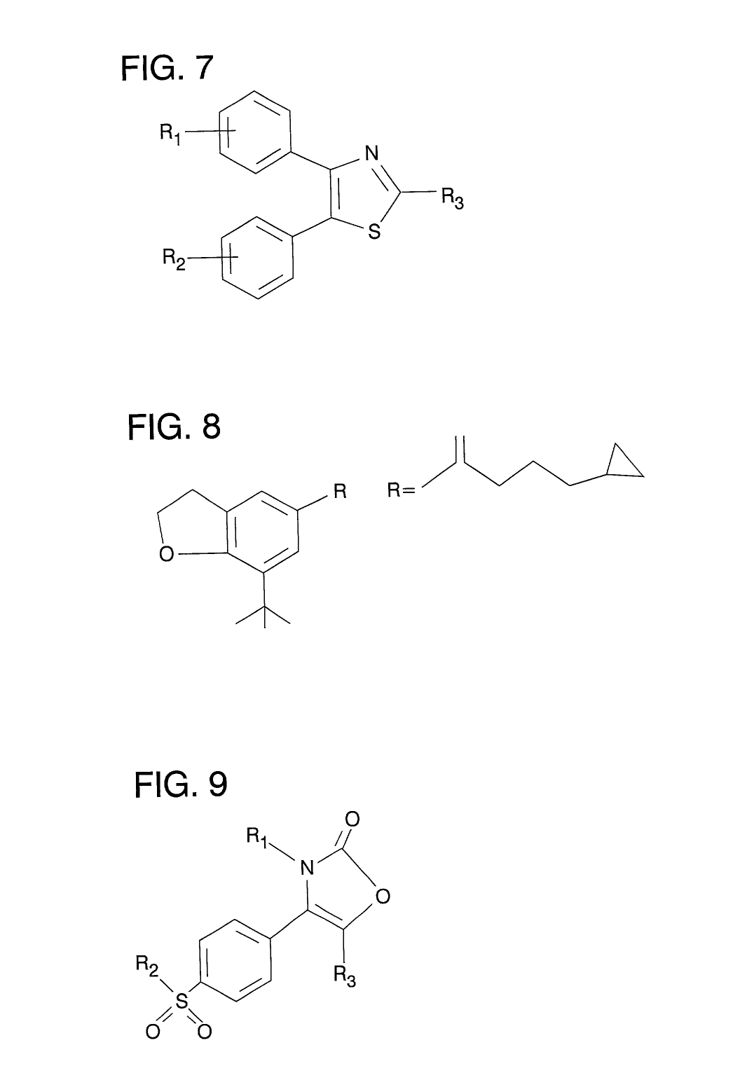

f) Sulfonamide-substituted 4,5-diaryl thiazoles (GD-Searle-Monsanto). Most selective and potent compound of this series: R₁=H—, R₂=4—SO₂NH₂, R₃=—CH₃ (See FIG. 7) (ED₅₀=2 mg/kg)

g) L-745,337 (5-methane sulfonamido-6-(2,4-difluorothiophenyl)-1-indanone (Merck Frosst) ID50=0.4 mg/kg.

h) LeFlunomide

i) Meloxicam: dose range 7.5 mg—15 Mg

j) L-778,736 (Merck Frost)

k) Flosulide: ID₅₀<0.1 mg/kg

l) SC 58236, SC-57666, SC-58125: (Searle)

m) L-745,337

n) O-(Acetoxyphenyl) hept-2-ynyl sulfide (APCs)

o) diclofenac/misoprostol (Arthrotec, Searle)

p) Flunixin Melamine

q) PK-138387 (Parke-Davis)

r) JTE-522: (4-(4-cyclohexyl-2-methyloxazol-5-yl)-2-Fluorobenzene Sulfonamide) IC₅₀: 15.1 nm in cellular systems

s) Nimesulide: 100 mg, twice daily

t) SC-57666: 1-[2-(4-Fluorophenyl) cyclopenten-1-yl]-4-(methylsulfonyl) benzene (Searle). From the family of 1,2-diarlycyclopentenes, ED₅₀ low mg/kg

u) 5-Keto-substituted 7-tert-butyl-2,3-dihydro-3,3-dimethyl benzofurans (Proctor & Gamble). Most promising isomer: R=CO(CH₂)₃-c—Pr; ED₅₀=6.6 mg/kg (See FIG. 8)

v) Etodolac: 300 mg and 600 mg daily dose.

w) MK966: 25 mg and 125 mg daily dose

x) 3,4-diaryloxazolones (Almirall Prodesfarma, Spain) (See FIG. 9) Most promising: (Similar or better than Celebrex) ED₅₀<2 mg/kg

| R₁ | R₂ | R₃ |
|---|---|---|
| C₆H₅ | NH₂ | H |
| 2-FC₆H₄ | NH₂ | H |
| 4-FC₆H₄ | NH₂ | H |

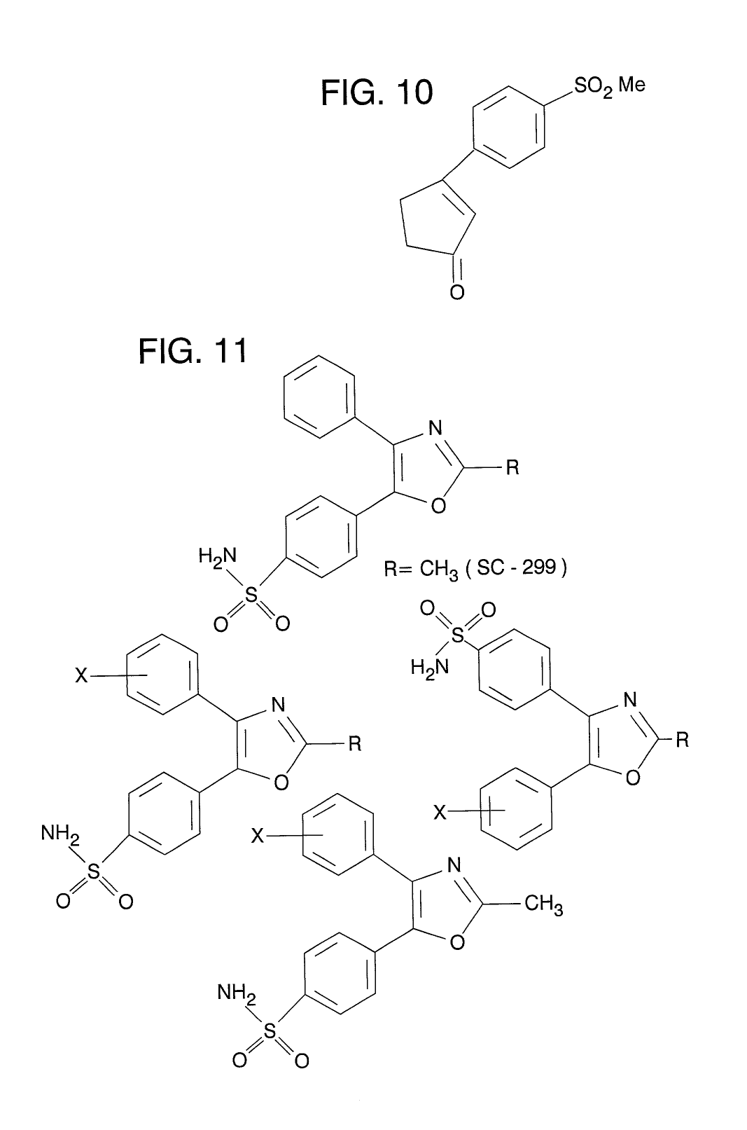

y) 2,3-Diarylcyclopentenones (Merck Frost) L-776,967 (ED₅₀: mg/kg) R=3,5-diflurophenyl L-784,506 (ED₅₀: 1.7 mg/kg R=3-pyridyl (See FIG. 10)

z) 4,5-Diaryloxazoles (G.D. Searle-Monsanto) (methyl sulfonyl or sulfonamido substituted) (See FIG. 11) where X is H,4Cl, 4—F, 4—Br or F; Y is H, 4—Cl, 4—Br, 3F, 4-OCH3, 3,4diCl, 3,4diF, or 3—F; and R is CH₃

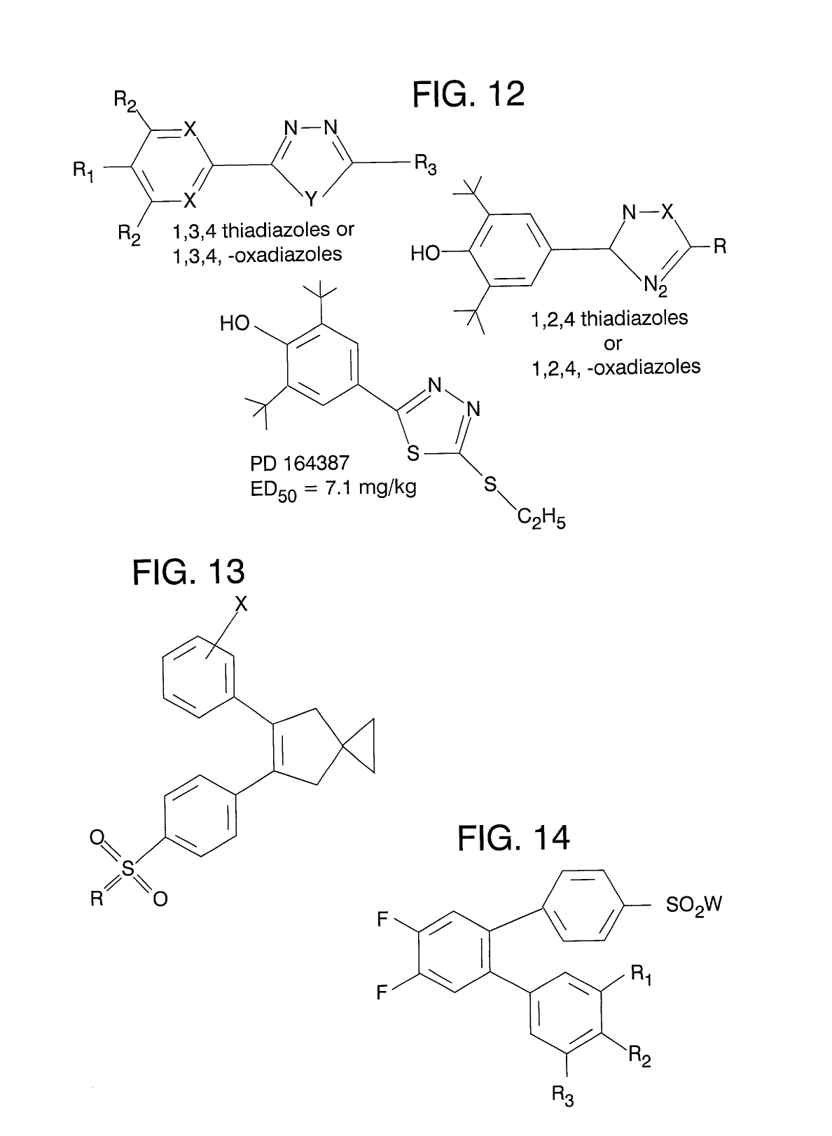

aa) Ditert-butyl phenols (1,3,4-and 1,2,4-thiadiazole series) (Parke-Davis) (See FIG. 12) where R₁, =OH; R₂=t-butyl, X=C, Y=S and R₃ is —S-Et (PD164387), ED₄₀=7.1 mg/kg

bb) SC 8076

cc) SC 8092

dd) CIP 28238

ee) 2acetoxyphenylalkylsulfides such as 2.acetoxyphenyl hept-2-ynyl sulfide, 2-acetoxyphenyl heptyl sulfide, and 2-acetoxyphenyl methyl sulfide

ff) Diarylspiro [2.4] heptenes (G.D. Searle/Monsanto) (See FIG. 13) where X is 3, 5-dichloro-4methoxy and R is —CH₃, ED₅₀=.52. Or when X=3-chloro-4-methoxy and R is NH₂, ED₅₀=0.21 mg/kg

gg) Terphenyl methyl sulfones and sulfonamides (Searle/Monsanto) 1,2-diaryl-4,5-difluorobenzene sulfonamides (See FIG. 14) where W is —CH₃ or —NH₂, R₁ is —H, R₂ is (CH3)2N— and R₃=Cl—

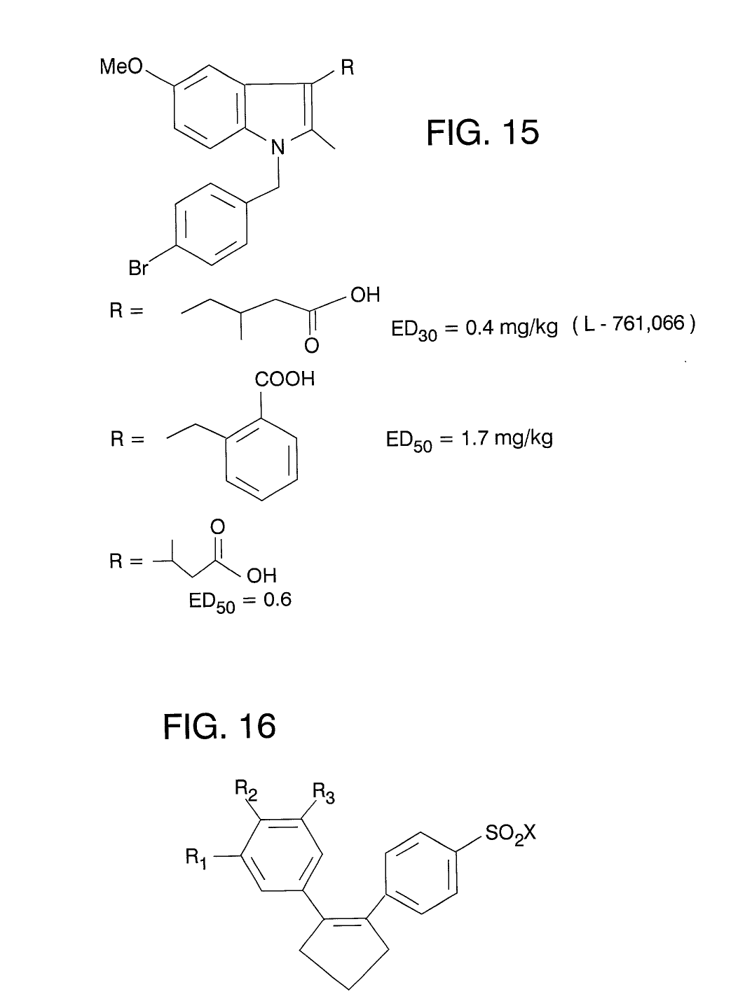

hh) Indolealkanoic acids (Merck-Frosst) (See FIG. 15)

ii) RS-75067

jj) 1,2-Diarylcyclopentenes (Searle/Monsanto) (See FIG. 16) where X=NH₂ or CH₃; R₁ is H, R₂ is F or Cl and R₃ is F or Cl (isomer of choice: R₁=H, R₂=F, R₃=CL, ED₅₀=0.16 mg/kg)

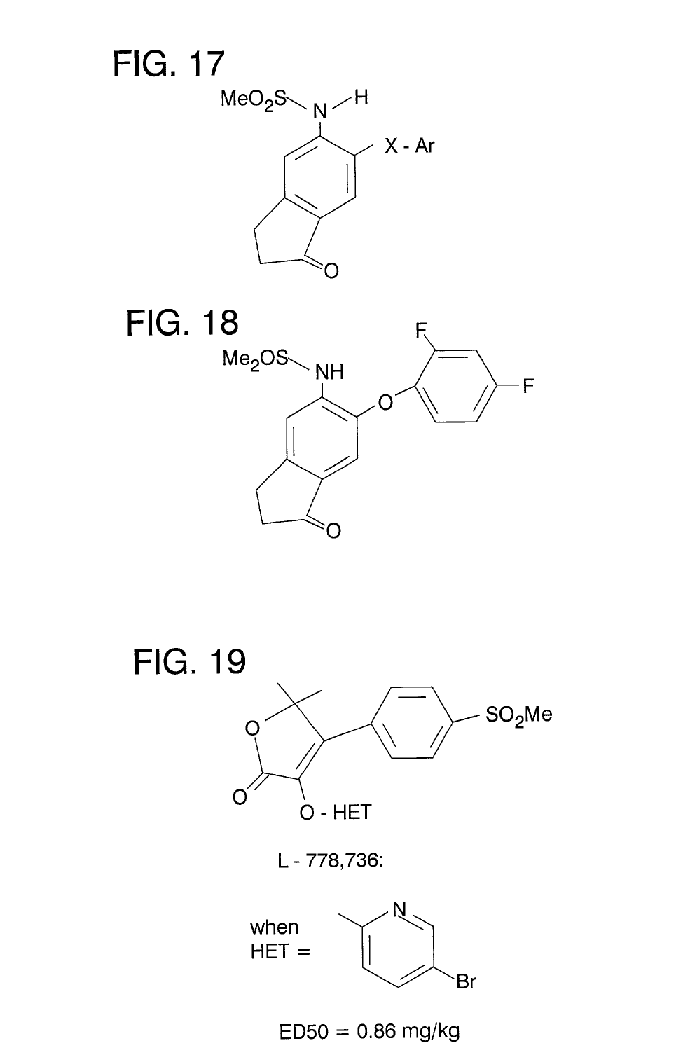

kk) 5-Methane sulfonamide-1-indanone derivatives (See FIG. 17) where X=S or O when X=S and pr=2,4 difluorophenyl (L-745,337) the ED₅₀=2mg/kg. FIG. 18 shows the isoprelated inhibitor: Flosulide.

ll) CGP 28238 (6-(2,4 difluorophenoxy)-5-methylsulfanoylamino-1-Antonina)

mm) BF 389

nn) T-614 (3-formylamino-7-methylsulfonylamino-6-phenoxy-4 H-1-benzopyran-4-one)

oo) L-745,337 (Merck-Frosst), 5-methanesulfonamide-6-(2,4-difluorothiophenyl)-1

indanone) ED₅₀=2mg/kg

pp) RWJ 63556 (R.W. Johnson Pharmaceutical Research Institute)

qq) (R,S)-Ketorolac; (S)-Ketorolac (Roche Bioscience)

rr) 3-Heteroaryloxy-4-phenyl-2(5H)-furanones (Merck Frosst); L-778,736, ED₅₀=0.86 mg/kg (see FIG. 19)

ss) terphenyl and the 2-pyridyl derivatives of methyl sulfones (Dupont)

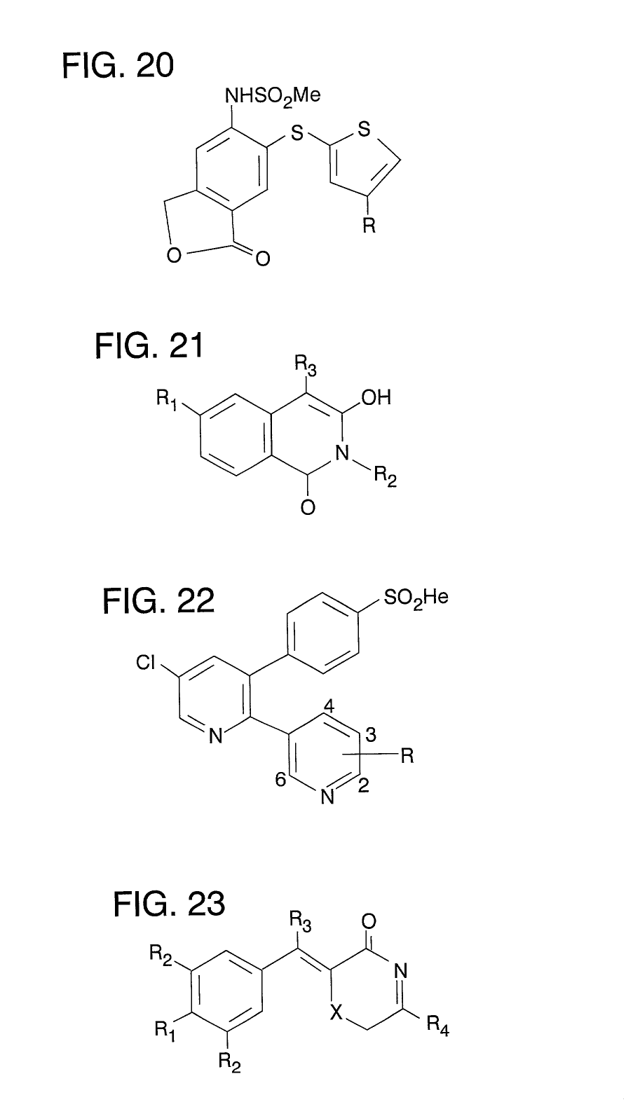

tt) substituted heterocyclic analogs in the Flosulide class (Merck Frosst) (See FIG. 20) where R=CH₂CH₃: 6(4-ethyl-2-thidyolylthio)-5-methanesulfonamido-3H-isobenzofuran-1-one

uu) 2-benzyl-4-sulfonyl-4-H-isoquinoline-1,3-diones (Boehringer Ingelheim Pharm.) (See FIG. 21) where R₁, is Cl, R₂ is CH₂ is CH₂Ph or CH₂ (13.4 difluoro-Ph) and R₃ is SO2-i-Pr with an ED₅₀=30mg/kg

vv) 2-pyridinyl-3-(4-methylsulfonyl) phenylpyridines (Merck Frosst), Isomer of choice: 5-Chloro-3(4-methylsulfonyl) phenyl-2-(2-methyl-5-pyridinyl) pyridine (L-791,456) ED₅₀: 0.6 mg/kg. (See FIG. 22) where R=2—Me

ww) Di-term-butyl phenols (thiazolone and oxazolone derivative) (Parks-Davis) FIG. 23 shows a representative compound where X=S; R₁=OH, R₂=t-Bu, R₃=H and R₄=NHOMe, ED₅₀=16 mg/kg.

xx) A 771726

### EXAMPLE 2

### Inhibitors of NF—-κB Activation

Protective Effect of Pyrrolidine Dithiocarbamate (PDTC) Against IDDM Onset.

PDTC is an established inhibitor ofthe transcription factor-κB (NF-κB). NF-κB is critically involved in the expression of several inflammatory mediators such as COX-2, inducible nitric oxide synthase (iNOS) and cytokines. It is now well-established that the activation of the transcription factor NF-κB is a crucial trigger of inflammatory responses including cytokines, iNOS, and COX-2 induction (13, 27, 40, 49, 50, 56). Exposure to pro-inflammatory stimuli, such as TNF-α or IL-1, results in the proteolytic destruction of the endogenous NF-κB inhibitor IκB (4, 13, 29, 40, 50), leading to the translocation of NF-κB to the nucleus, where it binds to DNA and sets in motion the transcription of target genes. Several studies have shown that NF-κB activation is involved in cytokine-mediated expression of iNOS in isolated rat and human islets and in RIN-m5F insulinoma cells (14, 18, 34, 35). Kwon et al. have demonstrated that NF-κB activation is involved in IL-1-induced PGE₂ formation by isolated rat islets (34).

The fact that NF-κB is a transcription factor for COX-2 gene expression taken together with the observation that PGE₂ causes the up-regulation of COX-2 MRNA, suggests that PGE₂ should, directly or indirectly, activate NF-κB. As a result, inhibition of PGE₂ formation should indirectly prevent the activation of NF-κB and all down-stream pathways that this transcription factor mediates, including INOS and COX-2 pathways. This notion is supported by the findings of Camandola et al. which demonstrated that PGE₂ activates NF-κB in U937 human promonocytic cells and in J774 macrophages (5). This observation has been confirmed by Dumais et al who have demonstrated that PGE₂ activated NF-κB, and consequently mediated HIV-1 transcription in T cells (16). Several other investigators have shown that arachidonic acid (COX-2 substrate and PGE₂ precursor) activates NF-κB in different cellular systems (44, 58, 70). According to our findings, phenyl N-tert-butylnitrone (PBN), which we have shown to protect against IDDM in low-dose STZ model (67), acts as an inhibitor of NF-κB activation in acute inflammation, and thus inhibits the induced expression of INOS and COX2 (33). Based on this hypothesis we have tested the possible effect of a known inhibitor of NF-κB activation, the compound PDTC against IDDM.

Method. CD-1 mice were divided into two groups (6 mice/group). Group 1 was treated with STZ (40 mg/Kg dissolved in cold sodium citrate buffer, pH 4.5) for 5 consecutive days. Group 2 was treated with STZ as described and received daily treatments of PDTC (150 mg/Kg, dissolved in physiological saline) starting from day 1 of STZ treatment. The PDTC dose was reduced to 100 mg/Kg at day 7 of the treatment. A total of 11 daily treatments of PDTC were given to the mice in this group (up to day 11 from the start of STZ treatment). Blood glucose levels were periodically determined in these mice.

Results. Mice treated with PDTC were protected from STZ-induced IDDM as shown in Table IV below. As seen in Table IV, STZ treated mice showed progressive hyperglycemia following the STZ treatment. At day 19 of treatment, the blood glucose level of this group was extremely high (mean value: 446.7 mg/dl). However, the PDTC treated mice showed only a steady and mild hyperglycemia. At day 19 of treatment, the blood glucose level of this group was found to be only mildly elevated (mean value: 199.6 mg/dl). Thus, inhibition of activation of NF-κB by means of PDTC administration was found protective against IDDM in mice. This observation is consistent with the hypothesis that COX-2 inhibition protects against IDDM through prevention of PGE₂-mediated activation of NF-κB.

TABLE IV
PDTC PROTECTS AGAINST STZ-INDUCED IDDM

| Day of Treatment | Treatment | Blood Glucose Level (mg/dl) |
|---|---|---|
| 11 | STZ | 249.0 ± 50.2* |
| | STZ + PDTC | 190.8 ± 23.2 |
| 19 | STZ | 446.7 ± 106.1** |
| | STZ + PDTC | 199.6 ± 35.6 |

*The difference between the blood glucose values of the STZ group vs. the STZ + PDTC group was statistically significant at both time points (*p value = 0.026, **p value < 0.001).

Other inhibitors of NF-κB activation, such as, e.g., N-tert-butylnitrone may be likewise used.

Whereas, the present invention has been described in relation to the drawings attached hereto, it should be understood that other and further modifications, apart from those shown or suggested herein, may be made within the spirit and scope of this invention.

### REFERENCES

The following and previously mentioned references are incorporated in pertinent part by reference herein for the reason cited.

1. Anderson G D, hauser S D, McGarity K L, Bremer M E, Isakson P C, Gregory S A (1996) J Clin Invest. 97, 2672-2679.

2. Baek S H, Yun S S, Kwon T K, Kim J R, Chang H W, Kwak J Y, Kim J H, Kwun K B (1999) Shock 12, 473-478.

3. Berg, J., Fellier, H., Christoph, T., Grarup, J., Stimmeder, D. (1999) Inflamm. Res. 48, 369-79.

4. Callejas N A, Casado M, Bosca L, Martin-Sanz P. (1999) Requirement of nuclear factor kappaB for the constitutive expression of nitric oxide synthase-2 and cyclooxygenase-2 in rat trophoblasts. J Cell Sci.112, 3147-3155

5. Camandola S, Leonarduzzi G, Musso T, Varesio L, Carini R, Scavazza A, Chiarpotto E, Baeuerle P A, Poli G (1996) Nuclear factor κB is activated by arachidonic acid but not by eicosapentaenoic acid. Biochem. Biophys. Res. Commun. 229: 643-647.

6. Campbell, I. L., Iscaro, A. Q., and Harrison, L. C. (1988) J. Immunol. 141, 2325-2329.

7. Campbell, I. L., Kay T. W. H., Oxbrow, L., and Harrison, L. C. (1991) J. Clin. Invest. 87,739-742.

8. Chen, B., Chen, Y., Lin, W. (1999) Immunology 97, 124-129.

9. Chen, C. C., Chiu, K. T., Sun, Y. T., Chen, W. C. (1999) J. Biol. Chem. 274, 31559-31564.

10. Corbett, J. A., Anwar, M., Shimizu, J., Frederick, K., Misko, T. P., McDaniel, M. L., Kanagawa, O., and Unanue, E. R. (1993) Proc. Natl. Acad. Sci. USA 90, 8992-8995.

11. Corbett, J. A., Kwon, G., Turk, J., McDaniel, M. L.(1993) Biochemistry 32, 13767-13770.

12. Dayer-Metroz MD, Wollheim C B, Seckinger P, Deyer J M (1989) J Autoimmun 2,163-171.

13. Delhase, M., Hayakawa, M., Chen, Y. & Karin, M. (1999). Positive and negative regulation of Iκβ kinase activity through IKKbeta subunit phosphorylation. Science 284, 309-313

14. de-Mello MAR, Flodstrom M, Eizirik DL (1996) Ebselen and cytokine-induced nitric oxide synthase expression in insulin-producing cells. Biochem. Pharmacol. 52: 1703-1709.

15. DeWitt, D. L., Meade, E. A., Smith, W. L. (1993) Am. J Med. 5 (Suppl 2A), 40S-44S.

16. Dumais N, Barbeau, Olivier M, Tremblay M J (1998) Prostaglandin E₂ up-regulates HIV-1 long terminal repeat-driven gene activity in T cells via NF-κB-dependent and independent signaling pathways. J Biol Chem. 273, 27306-27314

17. Flodstrom M, Tyrberg B, Eizirik D L, Sandler S.(1999) Diabetes 48, 706-713.

18. Flodstrom M, Welsh N, Eizirik D. L. (1996) Cytokines activate the nuclear factor κB (NF-κB) and induce nitric oxide production in human pancreatic islets. FEBS Lett. 385, 4-6

19. Futaki. N., Arai, I., hamasaka, Y., Takahashi, S., Higuchi, S., Otomo, S. (1993) J. Pharm.

Pharmacol. 45, 753-755.

20. Futaki. N., Takahashi, S., Yokoyama, M., Arai, I., Higuchi, S., Otomo, S. (1994) Prostaglandins 47, 55-59.

21. Ganda O P, Rossini A A, Like A A (1976) Studies on streptozotocin diabetes. Diabetes 25: 595-603

22. Goldenberg, M. M. (1999) Clin. Ther. 21, 1497-1513.

23. Herold K C, Bloch T N, Vezys V, Sun Q (1995) Diabetes induced with low doses of streptozotocin is mediated by V beta 8.2+T-cells. Diabetes 44: 354-9.

24. Hinson R M, Williams J A, Shacter E (1996) Natl. Acad. Sci. USA 93, 4885-4890.

25. Hughes H H, Easom R A, Wolf B A, Turk J, McDaniel M L(1989) Diabetes 38, 1251-1257.

26. Jobin, C., Morteau, O., Han, D. S., Balfour Sarter, R. (1998) Immunology 95, 537-543.

27. Jourd'heuil D, Morise Z, Conner E M, Grisham M B (1997) Oxidants, transcription factors, and intestinal inflammation. J. Clin. Gastroenterol. 25 Suppl 1: S61-72.

28. Kantwerk-Funke G, Burkart V, Kolb H (1991) Low dose streptozotocin causes stimulation of the immune system and anti-islet cytotoxicity in mice. Clin Exp Immunol 86:266-70

29. Karin, M. (1997). The IκB kinase complex (IκK) contains two kinase subunits, IκKα and IKKβ, necessary for I?B phosphorylation and NF-κB activation. Cell 91, 243-252

30. Kim Y T, Steinberg C (1984) Immunologic studies on the induction of diabetes in experimental animals. Cellularbasis forthe induction ofdiabetes by streptozotocin. Diabetes 33:771-7.

31. Kleeman R, Rothe H, Kolb-Bachofen V, Xie Q-W, Nathan C, Martin S, Kolb H. (1993) FEBSLett. 328, 9-12.

32. Kolb H, Kiesel U, Kroncke K D, Kolb-Bachofen V. (1991) Life Sci 49, PL213-217.

33. Kotake, Y. sang, H., Miyajima, T., Wallis, G. L.(1998) Biochim. Biophys. Acta 1448,77-84.

34. Kwon, G., Corbett, J. A., Hauser, S., Hill, J. R., Turk, J., McDaniel, M. L. (1998) Diabetes 47, 583-591.

35. Kwon G, Corbett J A, Rodi C P, Sullivan P, McDaniel M L (1995) Interleukin-1-β-induced nitric oxide synthase expression by rat pancreatic β-cells: Evidence for the involvement of nuclear factor κB in the signaling mechanism. Endocrinology 136, 4790-4795

36. Liang, Y. C., Huang, Y. T., Tsai S. H., Lin-Shiau, S. Y., Chen, C. F., Lin, J. K. (1999) Carcinogenesis 20, 1945-1952.

37. Like AA, Rossini AA (1976) Streptozotocin-induced pancreatic insulitis: New model for diabetes mellitus. Science 193: 415-417

38. Litherland, S. A., Xie, X. T., Huston, A. D., Waseerfall, C., Whittaker, D. S., She, J. X., Hofig, A., Dennis, M. A., Fuller, K., Cook, R., Schatz, D., Moldawer, L. L., Clare, Salzler, M. J. (1999) J. Clin. Invest. 104, 515-523.

39. Liu S F, Ye X, Malik A B (1999) Inhibition of NF-κB activation by pyrrolidine dithiocarbamate prevents In vivo expression of proinflammatory genes. Circulation 100: 1330-1337.

40. Lukic, M. L., Stosic-Grujicic, S., Ostojic, N., Chan, W. L., and Liew, F. Y. (1991) Biochem.

Biophys. Res. Commun. 178, 913-920.

41. Mandrup-Poulsen, T., Bendtzen, K., Dinarello, C. A., andNerup, J. (1987)J. Immunol. 139, 4077-4082.

42. Maziere C, Conte M A, Degonville J, Ali D, Maziere J C (1999) Cellular enrichment with polyunsaturated fatty acids induces an oxidative stress and activates the transcription factors AP1 and NFκB. Biochem. Biophys. Res. Commun. 265: 116-122.

43. McDaniel M L, Kwon G., Hill J R, Marshall C A, Corbett J A. (1996) Proc Soc Exp Biol Med. 211, 24-32.

44. Nagayama, M., Niwa, K., Nagayama, T., Ross, M. E., Iadecola, C. (1999) J. cereb. Blood Flow metab. 19, 1213-1219.

45. Nicoletti, F., Di Marco, R., Barcellinini, W., Magro, G. Schorlemmer, H. U., Kurrle, R., Lunetta, M., Grasso, S., Zaccone, P., and Meroni, P. (1994) Eur. J Immunol. 4, 1843-1847.

46. Nogawa WS, Forster C, Zhang F, Nagayama M, Ross M E, Iadecola C (1998) Proc. Natl Acad Sci USA 95, 10966-10971.

47. Pelletier, J. P., Lascau-Coman, V., Jovanovic, D., Fernandes, J. C., Manning, P., Connor, J. R., Currie, M. G., Martel-Pelletier, J. (1999) J. Rheumatol. 26, 2002-2014.

48. Pistritto G, Franzese O, Pozzoli G, Mancuso C, Tringali G, Preziosi P, Navarra P (1999) Bacterial lipopolysaccharide increases prostaglandin production by rat astrocytes via inducible cyclo-oxygenase: evidence for the involvement of nuclear factor κB. Biochem. Biophys. Res. Commun.263: 570-574.

49. Portanova J P, Zhang Y, Anderson G D, Hauser S D, Masferrer J L, Seibert K, Gregory S A, Isakson P C (1996) J Exp Med. 184, 883-891.

50. Posades, I., Terencio, M. C., Guillen, I., Ferrandiz, M. L., Coloma, J., Paya, M., Alcaraz, M. J. (2000) Naunyn Schmiedbergs Arch. Pharmacol. 361, 98-106.

51. Pukel, C., Baquerizo, H., and Rabinovitch, A. (1988) Diabetes 37, 133-136.

52. Rabinovitch A, Suarez-Pinzon W L, Sorensen O, Bleackley R C. (1996) Endocrinology 137, 2093-2099.

53. Reimers, J. I., Bjerre, U, Mandrup-Poulson, T., and Nerup, J. (1994) Cytokine 6, 512-520.

54. Rivest S. (1999) What is the cellular source of prostaglandins in the brain in response to systemic inflammation? Facts and controversies. Mol Psychiatry.4, 500-507

55. Romay, C., Ledon, N., Gonzalez, R. (1998) Inflamm. Res. 47, 334-338.

56. Rusyn I, Bradham C A, Cohn L, Schoonhoven R, Swenberg J A, Brenner D A, Thurman R G. (1999) Corn oil rapidly activates nuclear factor-εB in hepatic Kupffer cells by oxidant-dependent mechanisms. Carcinogenesis 20: 2095-2100.

57. Sandberg, J. -O., Anderson, A., Eizirik, D. L., and Sandberg, S. (1994) Biochem. Biophys. Res. Commun. 202, 543-548.

58. Sandler, S., Anderson, A., and Hellerstrom, C. (1987) Endocrinology 121, 1424-1431.

59. Schatz, D., Krischer, J., Home, G., Riley, W., Spillar, R., Silverstein, J., Winter, W., Muir, A. (1994) J. Clin. Invest. 93, 2403-2407.

60. Schneider E, Wohlrab F, Cossel L (1984) Morphologic-histochemical characterization of inflammatory cells in insulitis induced by multiple subdiabetogenic doses of streptozotocin in C57BI/KsJ mice. Biomed Biochim Acta 43:683-90

61. Seibert, K., Masferrer, J. L. (1994) Receptor 4, 17-23.

62. Serou, M. J., DeCoster M. A., Bazan, N. G. (1999) J. Neurosci. Res. 58, 593-598.

63. Slivka S R, Insel P A (1988) J. Biol. Chem. 263, 14640-14647.

64. Tabatabaie T, Kotake Y, Wallis G L, Jacob J M, Floyd R A (1997) Spin trapping agent phenyl N-tert-butylnitrone protects against the onset of drug-induced insulin-dependent diabetes mellitus. FEBS Lett. 407: 148-152.

65. Takamura T, kato I, Kimura N, Nakazawa T, Yonekura H, Takasawa S, Okamoto H. (1998) J Biol Chem 273, 2493-2496.

66. Timsit, J., Boitard, C., Bach, J. F. (1991) Ann. Biol. Clin. 49, 149-153.

67. Toborek M, Malecki A, Garrido R, Mattson M P, Hennig B, Young B (1999) Arachidonic acid-induced oxidative injury to cultured spinal cord neurons. J Neurochem. 73: 684-692.

68. Welsh M, welsh N, Bendtzen K, Mares J, Strandell E, Öberg C, Sandler S. (1995) Diabetologia 38, 53-160.

69. Yang, X., Ma, N., Szabolcs, M. J., Zhong, J., Athan, E., Sciacca, R.R., Michler, R. E., Anderson, G. D., Weise, J. F., Leahy, K. M., Gregory, S., Cannon, P. J. (2000) Circulation 101, 430-438.

70. Yuan C J, Mandal A K, Zhang Z, Mukherjee AB (2000)Cancer Res. 60, 1084-1091.

What is claimed is:

1. A method for prevention of insulin-dependent diabetes comprising administering an effective amount of a cyclooxygenase-2 inhibitor or repressor of NF-κB activation to an individual having a possible predisposition to or showing signs of development of Type 1 diabetes.

2. The method of claim 1 wherein COX-2 activity is decreased by the inhibition or the repression.

3. The method of claim 1 wherein the cyclooxygenase-2 inhibitor is NS-398.

4. The method of claim 1 wherein the repressor is PDTC.

5. The method of claim 1 wherein the therapeutic effective amount of cyclooxygenase-2 inhibitor is from about 2 mg/kg to about 10 mg/kg body weight.

6. The method of claim 1 wherein the effective amount of repressor is from about 25 to about 250 mg/kg/day.

7. The method of claim 1 where the individual's predisposition is determined by at least one of a history of Type 1 diabetes in an immediate family member and finding antibodies directed toward antigens of the individual's pancreatic beta cells.

8. A method of inhibiting allograft rejections comprising administration of an effective amount of a COX-2 inhibitor or repressor of NF-κB activation to an allograft recipient.

9. The method of claim 5 wherein the allograft is pancreas, kidney, liver or heart.

10. A method for prevention of insulin-dependent diabetes comprising administering an effective amount of NS-398 or PTC to an individual having a possible predisposition to or showing signs of development of Type 1 diabetes.

11. A method for prevention of insulin-dependent diabetes comprising administering an effective amount of NS-398 to an individual having a possible predisposition to or showing signs of development of Type 1 diabetes.

12. A method for prevention of insulin-dependent diabetes comprising administering an effective amount of PDTC to an individual having a possible predisposition to or showing signs of development of Type 1 diabetes.

13. The method of claim 10 where the individual's predisposition is determined by at least one of a history of Type 1 diabetes in an immediate family member and finding antibodies directed toward antigens of the individual's pancreatic beta cells.

14. An inhibitor of COX-2 activity or a repressor of NF-κB activation for use in the prevention of Type 1 diabetes or allograft rejection.

15. A method for prevention or treatment of diabetic complications comprising administering an effective amount of a cyclooxygenase-2 inhibitor or repression of NF-κB activation to an individual having Type 1 diabetes.

16. The method of claim 15 where the complications are nephropathy, retinopathy, neuropathy or cardiovascular disease.

17. The method of claim 15 wherein COX-2 activity is decreased by the inhibition or the repression.

18. The method of claim 15 wherein the cyclooxygenase-2 inhibitor is NS-398.

19. The method of claim 15 wherein the repression is PDTC.

20. The method of claim 15 wherein the therapeutic effective amount of cyclooxygenase-2 inhibitor is from about 2 mg/kg to about 10 mg/kg body weight.

21. The method of claim 15 wherein the effective amount of repression is from about 25 to about 250 mg/kg/day.

22. A method for prevention or treatment of diabetic complications comprising administering an effective amount of NS-398 or PDTC to an individual having Type 1 diabetes.

23. A method for prevention or treatment of diabetic complications comprising administering an effective amount of NS-398 to an individual having Type 1 diabetes.
